(19) United States
(12) Patent Application Publication
(10) Pub. No.: US 2004/0101929 A1
(43) Pub. Date: May 27, 2004

(54) Expressing mammalian protein complexes in fish

(76) Inventors: Chi-Yao Chang, Taipei (TW); Yu-Shen Lai, Taipei (TW); Joseph Abraham Christopher John, Taipei (TW)

Correspondence Address: Y. ROCKY TSAO, Fish & Richardson P.C., 225 Franklin Street, Boston, MA, 02110-2804, US

(21) Appl. No.: 10/302,570

(22) Filed: Nov. 22, 2002

(51) Int. Cl. A01K 67/027; C12P 21/02; C12N 5/06

(52) U.S. Cl. 435/69.1; 435/339; 435/320.1

(57) ABSTRACT

A fish cell expressing a mammalian protein complex having two peptide species. The cell has a first nucleic acid sequence encoding a first peptide species of the mammalian protein complex and a second nucleic acid sequence encoding a second peptide species of the mammalian protein complex. A multiplicity of peptides of the first peptide species and the second peptide species are expressed and at least one peptide of the first peptide species and at least one peptide of the second peptide species assembly to form a functional mammalian protein complex. The invention also discloses a method of making such a cell, and a cultured fish cell infected with a virus or its progeny for producing the virus.

### BACKGROUND

There is a need to generate fish that are capable of expressing mammalian protein complexes.

For example, infectious diseases are common on fish farms due to intensive fish farming that facilitates the transmission of pathogens in an aqueous environment. By binding to pathogens, antibodies inactivate them and therefore protect fish from infectious diseases. In teleost fish, only two types of antibodies, IgM and IgD, have been found. IgM, the major isotype, binds to and inactivates pathogens. However, the efficacy is not satisfactory due to relatively low binding affinity. Expressing mammalian antibodies in fish has several advantages in preventing the fish from infectious diseases. First, antibodies with high affinity can be made using the mammalian hybridoma technology. In addition, genes encoding the antibodies can be introduced into germ-line cells of fish, and transmitted to and expressed in the progenies of the fish. Further, the expressed antibodies can protect the fish progenies during their early stage of development, when the immune system has not fully developed.

Lorenzen reported expressing mammalian single-chain antibodies in fish cells (Nat. Biotechnol. 18:1177-1180, 2000). The single-chain antibodies each contain a single hybrid polypeptide chain having a constant region of an Ig kappa chain, and variable regions of heavy and light chains. As these single-chain antibodies are not stable, they only offer limited protection.

### SUMMARY

One aspect of the present invention features a fish cell expressing a mammalian protein complex having two peptide species. The fish cell contains a first nucleic acid sequence encoding a first peptide species of a mammalian protein complex, and a second nucleic acid sequence encoding a second peptide species of the mammalian protein complex. In the fish cell, a multiplicity of peptides of both peptide species are expressed, and at least one peptide of the first peptide species and at least one peptide of the second peptide species assembly to form a functional complex, i.e., a complex capable of performing the task of a naturally occurring protein complex.

The fish cell can be prepared from cells of different tissues (e.g., brain), of various fish such as yellow grouper. The above-mentioned protein complex consists of, e.g., one to four subunits of each of the two peptide species. An example of such a mammalian protein complex is a mammalian antibody, i.e., an immunoglobulin (Ig) having two heavy chains and two light chains, optionally containing a disulfide bond. In one embodiment, a functional antibody specifically binds to a microorganism, e.g., a virus, a bacterium, a protozoan, or a parasite. Another example of a mammalian protein complex is a mammalian signaling molecule, such as a cytokine or a growth factor. A signaling molecule binds to its receptor on a cell and triggers a cellular response. An interlukin, a cytokine, contains only two subunits of two different peptide species.

In another aspect, the present invention features a method of expressing in fish, e.g., a yellow grouper, one of the mammalian protein complexes described above. The method includes introducing into fish cells a eukaryotic expression cassette that contains two sequences encoding a first peptide species and a second peptide species, respectively, of the mammalian protein complex. Each sequence is operably linked to a transcription promoter in a manner that allows expression of a peptide encoded by the sequence. In the cells, a multiplicity of peptides of both peptide species and the second peptide species are expressed and at least one peptide of the first peptide species and at least one peptide of the second peptide species assembly to form the complex. The expression cassette can be introduced into a brain cell. It was unexpected that one can express a functional mammalian protein complex in a fish cell.

In yet another aspect, the present invention features a cultured fish cell (e.g., a brain cell) infected with a virus, or a progeny of the cultured fish cell. The cultured fish cell can be used as a resource for the virus. It can be made from various cell types of various fish species (e.g., a yellow grouper).

Other features, objects, and advantages of the invention will be apparent from the description and the claims.

### DETAILED DESCRIPTION

One can generate a fish cell that expresses a mammalian protein complex by introducing into the cell a eukaryotic expression cassette that contains two sequences. The sequences encode a first peptide species and a second peptide species, respectively, of the mammalian protein complex. Each sequence is operably linked to a transcription promoter in the cassette. The sequence can be operably linked to a common transcription promoter or to separate promoters.

Alternatively, one can introduce into the cell two expression cassettes, each containing a sequence encoding the first or the second peptide species. In all cases above, the cassette optionally contains other sequences, such as enhancers, polyadenylation signals, or sequences encoding selectable markers (e.g., an antibiotic resistance protein and green fluorescent protein).

A cassette can be introduced into a fish cell, either cultured in vitro or existing in a fish, via conventional transformation or transfection techniques, including a variety of art-recognized methods for introducing a foreign nucleic acid into a suitable cell, e.g., calcium phosphate or calcium chloride co-precipitation, DEAE-dextran-mediated transfection, lipofection, electroporation, or microinjection. The cassette can be introduced into cells in a fish using techniques described in, e.g., Lorenzen N. et al., Nat Biotechnol. 18:1177-80, 2000. The cells or fish are then tested for the expression or activity of the mammalian protein complex by the method described in the actual example provided below or by other methods well known in the art.

Examples of a mammalian complex include an antibody that consists of two heavy chains and two light chains. This antibody specifically binds to a pathogen such as a virus, e.g., nervous necrosis virus, iridovirus, infectious pancreatic necrosis virus, infectious hematopoietic necrosis virus, viral hemorrhagic septicemia virus, and lymphocystis disease virus, thereby protecting a fish cell or a fish from infection by the pathogen.

What antibody to be expressed in a fish depends on what pathogen to be protected from. To prepare a fish cell expressing a mammalian antibody, one uses a pathogen or an antigen from it to immunize a mammal, e.g., a mouse or a rat. Using the standard hybridma technology, one can generate hybridma cells producing monoclonal antibodies (MAbs) that specifically bind to the pathogen. Using cDNA library constructing and screening techniques, one can isolate cDNAs encoding the heavy and light chains of the specific MAbs. The isolated cDNAs can be cloned into an expression cassette and introduced into fish cells or fish as described above. Cells or fish that receive the cassette express the MAbs specifically against antigens of pathogens, e.g., a coat protein of a virus. The MAbs mask the coat proteins via which the virus attaches to and enters target fish cells, and therefore protect the fish from diseases caused by the virus. If the cassette is introduced into the germ-line cells of the fish, they can be transmitted to its progenies. As a result, the progenies also produce the MAbs and are protected from the virus. MAbs produced by fish cells of the invention contain all four chains of mammalian antibodies. They correctly fold and assembly, and are more resistant to proteolysis and more stable than a single chain antibodies mentioned above. It was unexpected that one can express a stable, functional mammalian antibody with all four chains in fish non-immune cells, e.g., brain cells.

Examples of a mammalian protein complex also include a signaling molecule, such as an interlukin, an inhibin or an activin, that consists of two peptide species. The genes encoding different peptide species of a signaling molecule can be cloned and introduced into fish cells or fish as described above. A mammalian signaling molecule expressed by the cells or fish can improve the immune response of the fish. Also, the fish cells or fish can be used to study the signal transduction pathway of the signaling molecule.

Also within the scope of the invention are a cultured fish cell and its progeny that produce a virus. One can make a cultured fish cell or its progeny by infecting a fish cell with a virus using the method described in the actual example below. A virus produced by the cultured cell can be collected from a supernatant of a cell culture or a lysate of the cell using methods well known in the art. The cultured fish cell can be used to propagate viruses for research and to preserve endangered virus species. Researchers have attempted to make cultured fish cell lines to produce virus. However, titers of virus produced were not appreciable and could not be maintained in the progenies of the cells. It was unexpected that virus could be produced with high titers from a cultured fish cell and its progenies.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### EXAMPLE

1. Yellow Grouper Brain Cells

Primary culture of grouper brain cells. A healthy yellow grouper weighing 50 g was obtained from a fish farm in southern Taiwan. It was anaesthetized in iced water, dipped in 5% chlorex for 5 minutes and wiped with 70% alcohol. The brain of the fish was removed and washed three times in antibiotic medium containing, Leibovitz's L-15 (L-15), 400 IU ml⁻penicillin and 400 μg ml⁻¹ streptomycin. The brain then was minced thoroughly with scissors and transferred to a 60 mm diameter Nunc tissue culture disc for trypsinization using 10 ml 0.25% trypsin solution (0.25% trypsin and 0.2% EDTA in PBS). The resultant mixture was gently agitated using a magnetic stirrer at 4° C. for 1 hour. After undigested tissue pieces settled down, supernatant containing grouper brain (GB) cells was transferred into a tube and mixed with an equal volume of complete medium, i.e., L-15, 15% fetal bovine serum (FBS, Hyclone Laboratories, Logan, Utah, USA), 100 IU ml⁻¹ penicillin and 100 μg ml⁻¹ streptomycin, to inactivate the trypsin. The cells were centrifuged at 200×g for 10 minutes, and were resuspended in a fresh complete medium. 4×10⁵ cells were then seeded into a 25 μm² tissue culture flask containing 4 ml of L-15 with 15% FBS and grown at 28° C.

Subculture and maintenance of GB cells. A confluent primary cell culture was trypsinized and the subcultures were grown as described above. During the first 10 passages, the cells were subcultured every 8 days in a medium consisting of 50% fresh L15-10% FBS medium and 50% conditioned medium. For cultures between passage 10 and passage 41, the cells were subcultured in fresh L15-10% FBS every 7 days. For cells that have been cultured for more than 41 passages, they were subcultured in L15-10% FBS every 5 days. Cells that had been subcultured for more than 80 times were maintained in L-15-5% FBS.

A GB cell line was established from the primary GB cells using subculturing as described above. The cells had morphology similar to that of fibroblast and epithelial cells. Cells with similar morphology had also been found in grouper kidneys, livers, and fins.

Storage of GB cells. A 2-day old subculture of grouper brain cells was harvested by trypsinization and centrifugation as described above. The resultant cell pellet was resuspended at a density of 10⁶ cells ml⁻¹ in a medium containing 10% FBS and 10% dimethyl sulphoxide (DMSO). The cells were dispensed into 2 ml plastic ampoules, and kept at −20° C. for 4 hours and then at −75° C. overnight before being transferred into a liquid nitrogen tank (−196° C.). Frozen cells could be recovered from the storage by thawing in a 37° C. water bath. Following removal of the freezing medium by centrifugation, cells were resuspended in L-15-10% FBS. The viability of the cells was examined using trypan blue staining and a haemocytometer. After being stored in liquid nitrogen for more than one year, the cells had 95% viability and were genetically stable.

Growth of GB cells. To determine the optimum growth temperature, duplicate cultures of 10⁵ GB cells were grown at 20, 24, 28, 32 or 36° C. The number of cells in duplicate flasks at each temperature was recorded every day for 5 days. The GB cells could grow at temperatures between 24 and 36° C. The cells grew optimally at 32° C., and did not grow at 20° C. or below.

The effect of different concentration of FBS (2%, 5%, 10%, 15%, or 20%) on GB cell growth was assessed at 28° C. The GB cells grew rapidly in a media containing 10 to 20% FBS, but considerably slower in a medium with 5% FBS. No cell growth was observed in a medium with 2% or less FBS.

Chromosome analysis. GB cells of passage 60 were used to make a chromosomal preparation. One day after subculturing, the GB cells were arrested at metaphase by incubating with 0.2-μg ml⁻¹ of colcemid (Sigma, St. Louis, Mo., USA) for 2 hours at 28° C. After harvesting by centrifugation (70×g, 5 minutes), the cells were suspended and kept in a hypotonic solution containing 0.5% KCl for 10 minutes before being fixed in a methanol: acetic acid (3:1) solution. Slides were prepared using the drop-splash method as described in, e.g., Culture of animal cells. In: A Manual of Basic Technique, 3rd ed. (ed. by R. I. Freshney), Wiley-Liss, New York, 387-389. After being stained with 5% Giemsa (EMS, Fort Washington, Pa., USA) for 10 minutes, chromosomes of the cells were observed and counted under a Leica DMRE microscope (Leica Mikroskopie und Systeme GmbH, Wetzlar, Germany). Chromosomes of GB cells were very homogeneous: all chromosomes were approximately the same size and mostly metacentric. The number of chromosomes in a GB cell ranged from 20 to 50, and had a bimodal distribution with modes of 22-42.

2. Diseased Fish

Healthy and moribund yellow grouper larvae (1-2 cm in length) were obtained from a hatchery in southern Taiwan. The symptoms of yellow grouper larvae infected with nervous necrosis virus (NNV) included abnormal swimming behavior.

The fish were fixed in neutral phosphate-buffered 10% formalin, dehydrated through a graded ethanol series and embedded in paraffin. Five-micrometer sections were prepared, dewaxed in xylene, rehydrated through a graded ethanol series, rinsed in distilled water, and stained with haematoxylin and eosin for microscopic examination.

In sections of healthy fish, no vacuolated lesions was found, whereas all diseased fish had degenerated and vacuolated lesions in brains, eyes and spinal cords. In the brains, vacuolation was most conspicuous in the gray matter. In the eyes, the extent of vacuolation of retinal tissue varied with the layers. Large vacuoles were detected in the bipolar nuclear layer and the neurons of the ganglionic layer. Occasionally, scattered small vacuoles were observed in rod and cone cells and the bipolar neurons. In the spinal cords, vacuolation was seen in all parts. The vacuolation observed in brains, retinas and spinal cords were very similar to those seen in the larvae and juvenile stages of other cultured fish species having viral nervous necrosis (VNN) (see e.g., Glazebrook J. et al., J. Fish Dis. 13: 245-249, 1990 and Chi S. et al., J Fish Dis. 20: 185-193, 1997.

3. Viruses

Viral susceptibility of GB cells. Diseased yellow grouper larvae were cut into small pieces and ground using a mortar and pestle. The resultant homogenate was mixed with a LI 15 medium, and was centrifuged at 8000×g for 10 minutes. The supernatant was filtered through a 0.2-μm membrane. Ten microlitres of the filtrate was inoculated into a 25-cm² Nunc culture flask containing an 80% confluent monolayer of GB cells prepared 24 hours before the inoculation. The cells were incubated at 28° C. for 7 days. During this period, rounded, granular and refractile cells, i.e., Cytopathology effect (CPE), could be found in some areas. The supernatant from cells that showed CPE was filtered through a 0.2-μm membrane and used for virus susceptibility studies.

GB cells (10⁵ cells ml⁻¹) were seeded into 24-well tissue culture plates and incubated at 28° C. until 80% confluence. Following a wash using PBS, the supernatant described above was inoculated into the wells at a multiplicity of infection (MOI) of 0.01 or at a series of 10×dilutions. The cells were cultured for 7 days at 28° C., and monitored for the CPE under an inverted microscope daily. Within 3 day after the inoculation, viral infection was detected in GB cells. Initially, the CPE could be found in some areas. During the next 3-4 days, the CPE spread throughout the cell culture, and cells degenerated and detached from the culturing dish. 8 days after the inoculation, the cells completely disintegrated and yielded virus-containing supernatant with a titre of 10^{8.5} TCID₅₀ mL⁻¹ (Tissue Culture 50% Infective Dose) determined according to the method described in Reed and Muench (1938; Amer. J Hygiene, 27: 493-497).

Electron microscopy. Three days after the inoculation, GB cells were harvested by centrifugation at 200×g for 5 minutes. The cells were fixed in a 2.5% glutaraldehyde-0.1M cacodylate buffer at 4° C. overnight and postfixed in 1% osmium tetroxide at 4° C. for 2 hours. The cells were washed in a cacodylate buffer, dehydrated in graded acetone solutions and embedded in Spur's low-viscosity resin. Silver-to-gold sections were cut on a diamond knife using a Reichert-Jung Ultracut E ultramicrotome (Nussloch, Germany). After being stained with 2% uranyl acetate (EMS, Fort Washington, Pa., USA) in distilled water for 1 minute, the sections were examined under a JEOL JEM 2000 EXII transmission electron microscope and photographed.

Aggregates of virus particles were found in the cytoplasm of diseased brain cells. These aggregates destroyed all cellular organelles. In non-infected cells, no virus particles were observed, and the nuclei and cellular organelles were intact. This is consistent with RT-PCR results showing no virus specific products from the nucleic acid extract prepared from normal cells.

Effect of temperature on yellow grouper NNV replication in GB cells. To determine the optimal temperature for NNV replication, GB cells (10⁵ ml⁻¹) were seeded into each well of 24-well tissue culture plates and incubated at 28° C. until cell cultures reached 80% confluence. The medium in each well was removed, and 1 ml virus-containing supernatant (titre 10^{8.5}) was inoculated into each well at a series of 10× dilution. The culture plates were incubated at 20, 24, 28, 32 or 36° C. After 7 days, the CPE was monitored described above. Levels of viral replication were different in yellow grouper NNV (YGNNV)-infected cells at different temperatures. The highest replication rate was observed at 28° C. (titre 10^{8.5}). The replication rate was reduced at 32° C. or 24° C., and significantly reduced at 20° C. or 36° C. These results are consistent with the fact that VNN —associated mass mortalities have been found in hatchery-reared grouper when the water temperature is around 28-32° C. (see, e.g., Breton A. et al., J Fish Dis. 20:145-151, 1997). Thus, the temperature plays an important role in YGNNV replication.

Purification of YGNNV. The supernatant from YGNNV-infected GB cells was filtered through a 0.2-μm membrane and inoculated onto GB cells grown in 175-cm² flasks at an MOI of 0.01. The virus was allowed to multiply at 28° C. for 7 days. The culture medium along with the adherent cells was collected in 250-ml Nalgene tubes. After centrifugation at 2000×g at 4° C. for 30 minutes (Beckman J2-21, JA 14 rotor, Spinco Division, Beckman Instruments Inc., Palo Alto, Calif., USA), the supernatant (containing free virus particles) and pellet (containing cell-associated virus) were collected separately.

The supernatant was adjusted so that it contained 2.2% (w/v) NaCl and 5% (w/v) polyethylene glycol (PEG, MW 8000, Sigma, St. Louis, Mo.). This mixture was stirred at 4° C. overnight before centrifuging at 3900×g at 4° C. for 1 hour. The resultant pellet was resuspended in 2 ml TNE buffer (50 mM Tris-HCl pH 7.3, 1 mM EDTA, 100 mM NaCl), extracted by mixing with an equal volume of Freon (1,1,2-trichlorotrifluoroethane, Sigma, St. Louis, Mo.), and centrifuging at 1800×g for 10 minutes. After this centrifuging, the supernatant was collected.

The cell-associated virus-containing pellet described above was suspended in 2 ml TNE buffer, and stored at 4° C. or extracted using Freon as described above.

The Freon-extracted samples were combined and laid on a three-step cesium chloride gradient (4 ml 40%, 3 ml 30%, 2 ml 20%) and centrifuged at 130,000×g at 4° C. for 16 hours (Hitachi SCP 85H2, RPS40T rotor, Hitachi Koki Co. Ltd., Minato-Ku, Tokyo, Japan). The virus-containing fraction was withdrawn using the method described in, e.g., Lai et al., J Fish Diseases 24:299-309, 2001. The fraction was then mixed with cesium chloride-TNE buffer so that the density and volume were 1.34 g ml-1 and 5 ml respectively. This mixture was centrifuged at 170,000×g for 16 hours at 4° C. using a Hitachi 55P-72 ultracentrifuge and a SW55T rotor. The virus band was collected and diluted using 5 ml TNE buffer before centrifuging at 230,000×g at 4° C. for 2 hours. The resultant purified virus pellet was resuspended in 1 ml TNE buffer. This purification procedure consistently yielded more than 3 mg of protein from twenty 175 cm²-flasks of virus-infected GB cells.

The purified viruses were examined by electron microscopy. 20 μl purified virus was placed on a 300-mesh grid and stained with 2% phospho-tungstic acid (pH 7.2). The virus was then observed under a Hitachi H-7000 transmission electron microscope and photographed. The virus exhibited a hexagonal shape with a diameter of 25-30 nm. This shape is common among fish nodaviruses, see, e.g., Yoshikoshi K. et al., J Fish Dis. 13:69-77, 1990, and Chi S. et al., J Fish Dis. 20: 185-193, 1997.

Extraction of viral RNA. A Diseased fish (100 mg) was frozen in liquid nitrogen and homogenized in 2 ml RNA isolation solvent (RNAzol B, TEL-TEST, USA) followed by mixing with 0.2 ml chloroform. The mixture was shaken vigorously for 15 seconds and held on ice or at 4° C. for 5 minutes. After extraction using the phenol and chloroform-isoamyl alcohol method, total nucleic acid was precipitated by isopropanol and then resuspended in diethyl pyrocarbonate-treated water. The purified nucleic acids were quantified using a Hitachi spectrophotometer model U-2000 (Hitachi Koki Co. Ltd., Minato-Ku, Tokyo, Japan).

Reverse transcription-PCR amplification. A set of two forward primers, F1: 5′-GGA ATT CCA TAT GGG ATT TGG ACG TGC GAC CAA-3′ (SEQ ID NO:1) and F2: 5′-GGA ATT CCA TAT GCG TGT CAG TCA TGT GTC GCT-3′ (SEQ ID NO:2), and two reverse primers, R1: 5′-CCG CTC GAG GTT TGC GGG GCA CAT TGG-3′ (SEQ ID NO:3) and R2: 5′-CCG CTC GAG CGA GTC AAC ACG GGT GAA GA-3′ (SEQ ID NO:4), were designed based on the sequence of RNA2 of a striped jack nervous necrosis virus (Nishizawa, T. et al., J. Gen. Virol. 76: 1563-1569, 1995) and were used for PCR amplification of three different target regions T2 (F1-R2: 892 bp), T4 (F2-R2: 443 bp) and T5 (F1-R1: 238 bp). The primers were synthesized by Quality Systems, Taiwan.

Five micrograms of RNA sample was preheated at 65° C. for 5 minutes and mixed with 70 μl of reverse transcription mixture containing 50 mM Tris-HCl, pH 8.3, 75 mM KCl, 3 mM MgCl₂, 1 mM of methylmercuric hydroxide, 7 mM β-mercaptoethanol, 3.8 μM of R2 primer, 1 mM of each dNTP, 40U of ribonuclease inhibitor, and 75U of Moloney murine leukaemia virus reverse transcriptase (Stratagene, La Jolla, Calif., USA). Reverse transcription was then carried out to prepare cDNA by incubating the mixture at 37° C. for 1 hour.

Following the reverse transciption, 2 μl of cDNA was added to a PCR mixture containing 2 μM forward primer, 2 μM reverse primer, 2.5 μM of each dNTP and 2.5U Taq DNA polymerase (Viogene, Taipei, Taiwan) to a final volume of 50 μl PCR buffer (15 mM Tris-HCl, pH 8.0, 50 mM KCl, 2 mM MgCl₂). PCR amplification was performed in an automatic thermal cycler (Perkin-Elmer 480, PE Biosystems, Foster City, Calif., USA) using following reaction condition: one cycle of incubation at 95° C. for 10 minutes, 35 cycles of incubation at 95° C. for 40 seconds/55° C. for 40 seconds/72° C. for 40 seconds, and one cycle of incubation at 72° C. for 10 minutes. After amplification, the PCR products were electrophoresed on a 1.2% agarose in a TAE buffer (40 mM Tris acetate, pH 8.3, 1 mM EDTA) gel and stained with ethidium bromide.

The sizes of target regions amplified from F1 to R2, from F2 to R2, and from F1 to R1 were 892 bp (T2), 443 bp (T4), and 238 bp (T5), respectively.

Cloning and sequencing of PCR products. Each of the forward primers (F1 and F2) contains an Nde I site, and each of the reverse primers (R1 and R2) contains an Xho I site. After digestion using Nde I and Xho I, the PCR products were ligated into the pET 20b (+) vector (Novagen, Darmstadt, Germany). The resultant recombinant plasmids were propagated in E. coli JM 109 cells.

PCR products of the T4 and T5 regions were sequenced using the F1, F2, R1, or R2 primers. The ends of the T2 region were sequenced using the F1 and R2 primers. Based on sequencing results, more primers were designed to sequence the full length of the T2 region. The nucleotide sequence of the plasmid insert was determined using T7 promoter and T7 terminator primers. All of the sequencing reactions were performed using Big Dye™ terminators and ABI PRISM 377 DNA auto-sequencer (PE Biosystems, Foster City, Calif., USA). The sequence of YGNNV T2 region was submitted to the GenBank database and assigned a GenBank Accession NO. AF 283554.

4. Antibodies

Preparation of monoclonal antibodies. BALB/cByJ mice were purchased from the National Laboratory Animal Breeding and Research Center, National Science Council, Taiwan. Monoclonal antibodies (MAbs) were generated using methods described in Harlow E. et al., Antibodies: A Laboratory Manual, Cold Spring Harbor Laboratory Press, Cold Spring Harbor, N.Y., 1988. Briefly, four 8-week-old mice were immunized with the YGNNV by an intraperitoneal injection with 100 μg of purified virus particles that was emulsified with an equal volume of Freund's complete adjuvant. After 14 days, a booster injection of a similar dosage that was prepared in Freund's incomplete adjuvant was given as described above. Two similar injections were administered at 2-week intervals. Two days after the last injection, spleen cells were collected from the mouse and fused with mouse myeloma cells (NS-1) at a ratio of 5:1 using 50% polyethylene glycol (MW 1500). Fused cells were resuspended in 15 ml of solution A (10 ml FBS, 2 ml 50×HAT, 10^{8.2} thymocytes in 3 ml of Iscove's Modified Dulbecco's Medium (IMDM: Gibco BRL, Life Technologies, Grand Island, N.Y., USA) and mixed with 25 ml of 2% methylcellulose in NIMDM. Seven to 10 days after fusion, the colonies were isolated and cultured in 96-well culturing plates. The supernatant from each well was screened for the presence of specific antibodies by an enzyme linked immunosorbent assay (ELISA).

Enzyme linked immunosorbent assay. Each well of Nunc-ELISA plates was coated with 100 μl (1 μg μl⁻¹) of purified YGNNV virus diluted in PBS containing 0.05% NaN₃ at 4° C. overnight. The wells were then rinsed three times with PBS and blocked with PBS containing 5% (w/v) skimmed milk at 37° C. for 2 hours. After three washes with PBS-TWEEN (PBS containing 0.05% (v/v) TWEEN20), 100 μl of control medium or a 1:100, 1:1000, 1:10,000, or 1:100,000 dilution of supernatant collected from each well where the fused cells grew was added to each well of the ELISA plates. The plates were incubated at 37° C. for 2 hours. After the plates were rinsed three times with PBS-TWEEN, 100 μl of 1:5000-diluted alkaline phosphatase-conjugated goat anti-mouse immunoglobulins (Pierce, Rockford, Ill., USA) was added to each well and incubated at 37° C. for 1 hour. After three washes with PBS-TWEEN, the activity of the alkaline phosphatase was visualized using 100 μl of 50% chromogenic substrate (pNPP: p-nitrophenyl phosphate, disodium salt) diluted in PBS-TWEEN. After a 30-min incubation in the dark, a yellow color developed in some wells. The optical density of solution in each well was read at 405 nm using an ELISA plate reader (CERES UV900 HDI, Bio-tek Instruments, USA). Clones with optical density values larger than 0.5 were positive clones. The supernatant of each of 1500 hybridoma clones was tested, and 43 clones were identified producing specific antibodies against YGNNV.

Neutralization of YGNNV in vitro. One milliliter of the supernatant from each hybridoma clone or the mock medium (control) was incubated with an equal volume of 10 fold dilutions of YGNNV (titre 10^{8.5}) in serum-free IMDM at room temperature for 1 hour with gentle shaking. The mixture was then added onto the 80% confluent monolayer of GB cell cultured in 24-well plates and incubated at room temperature for 30 minutes. The mixture in each well was then replaced with 1 ml fresh L-15-2% FBS medium before the culture plates were incubated at 28° C. for 7 days. The neutralizing activity of each MAb was determined by observation of the CPE, and the neutralization titre was calculated using the TCID₅₀ method as described above. The neutralization index (NI) was calculated using the method of Mahy et al., Neutralization. In: Virology Methods Manual (ed. by B. W. J. Mahy & H. O. Kangro), pp. 108-109. Academic Press, London, 1996. The difference between the titers of control and MAb treated samples was expressed as a neutralization index (NI), which was calculated using Formula I:

NI=log₁₀(TCID_{50 ∂mL}⁻¹ of the control sample)−log₁₀(TCID₅₀ mL⁻of the MAb-treated sample)

The supernatants of the 43 positive clones were examined. All of these clones possessed neutralizing activity, reducing the titre from 10^{8.5} (TCID₅₀ mL⁻¹) to the range between 10^{7.5} and 10² (TCID₅₀ mL⁻¹). MAbs produced by the 43 clones were grouped according to their NI values. Ten of them, RG-M3, RG-M18, RG-M30, RG-M33, RG-M41, RG-M47, RG-M50, RG-M51, RG-M56 and RG-M69, had NI values between 6.5 and 4.5. These antibodies are useful in protecting fish and in serological tests to diagnose infectious diseases in fish.

Isotype determination. MAb classes and subclasses were determined using a mouse MAb isotyping kit (ZYMED, San Francisco, Calif., USA). Alternatively, the classes and subclasses were determined by RT-PCR using primer sets specific for different antibody chains. The isotypes of 10 MAbs mentioned above were IgG with K light chains.

SDS-PAGE and Western blot analysis of the viral polypeptides. 1 μg of virus particles was separated by SDS-PAGE using the method of Laemmli et al., Nature 227: 680-685, 1970. The separated proteins were stained with Coomassie brilliant blue R-250. For Western blot analysis the proteins were electrotransferred onto a PVDF membrane (Amersham, Life Sciences, Buckinghamshire, England) using the method of Towbin et al, Proc. Nat. Acad. Sci., USA 76: 4350-4354, 1979. The membrane was washed with PBS-TWEEN followed by blocking in 5% skimmed milk overnight at 4° C. The membrane was then incubated with MAb (1:50) for 2 hours at room temperature, washed with PBS, and then incubated with alkaline phosphatase-conjugated goat anti-mouse immunoglobulins. Finally, proteins bound to by the antibodies were visualized using 5-bromo-4-chloro-3-indolyl phosphate and 4-nitro-blue tetrazolium chloride. The Coomassie brilliant blue staining revealed two major bands with molecular weights of 110 kDa (RNA1) and 42 kDa (RNA2), respectively. The 42 kDa protein, corresponding to the coat protein of nodavirus (MW 42 kDa), was recognized by all the 10 MAbs.

Virus coat protein synthesis in infected GB cells. Five 175-cm² flasks of GB cells were inoculated with YGNNV at an MOI of 1.0 and allowed to multiply at 28° C. At 6, 12, 18, 24 and 36 hours post-inoculation, the cells were collected by centrifugation at 70×g for 5 minutes. The cell pellets were lysed in 50 μl of sample buffer (100 mM Tris-HCl buffer, pH 6.8, 4% SDS, 0.07% β-mercaptoethanol, 20% glycerol, and 0.2% bromophenol blue). All samples were heated at 100° C. for 5 minutes, cooled to room temperature, and electrophoresed on 12% SDS-PAGE. The proteins were stained with Coomassie brilliant blue R-250 or electrotransferred to a PVDF membrane for Western analysis using RG-M56 MAbs. Western blot revealed that the 42 kDa protein was detected at 12 hours post-infection, and its level steadily increased afterwards.

Immunohistochemistry. Viruses were immuno-localized using the RG-M56 MAbs and the avidin-biotin-peroxidase complex technique (Vectastain® ABC kit, Vector Laboratories, Burlingame, Calif., USA). Diseased or healthy fish were fixed in neutral phosphate-buffered 10% formalin, dehydrated through a graded ethanol series, and embedded in paraffin. 3-μm sections were prepared, deparaffinized at 60° C. for 30 minutes, washed in three xylene baths, rehydrated through an ethanol series (100, 95, 90, 80 and 50%), and rinsed in distilled water. The sections were then treated with 0.1% trypsin and 0.3% hydrogen peroxide. Non-specific antibody binding sites were blocked using a normal serum provided with the Vectastain™ ABC kit. After rinsing with PBS, RG-M56 was added onto each section and incubated at 40° C. for 20 minutes. After a 5-min wash with PBS, secondary antibody was added and incubated at 40° C. for 10 minutes. After washing with PBS, 3,3′-diaminobenzidine (DAB) was added, and the color was allowed to develop for 5 minutes. The sections were washed in distilled water, counterstained with methyl green (Vector Laboratories, Burlingame, Calif., USA) and mounted for light microscopy examination.

The immunohistochemical examination revealed that lesions in diseased fish tissues were co-localized with specific immunostaining. In the brain of diseased fish, immunostaining was found in the granular layer of the optic tectem, telencephalon, cerebellum and medulla oblongata. In the retina, positive staining was seen mostly in cells of the bipolar and ganglionic layer. In the spinal cord, immunostaining was seen in all major parts. Neither lesions nor immunostaining were found in other tissues, e.g. gill or kidney, of the diseased fish.

It has been suggested that, from the skin, viruses enter the medial spinal cord through afferent nerves. From here, infection spreads anteriorly to the brain and retina, and posteriorly to the rest of the spinal cord (Nguyen et al., Dis. Aquat. Org. 24: 99-105, 1996). The immunohistochemistry staining could be observed only in retina, brain and spinal cord that have vacuolation. This indicates that the vacuoles are caused directly by the viral cytopathogenicity. 5. Expressing mammalian antibodies in fish cells and immunoprotection

Library construction and probe preparation. mRNA was isolated from 10⁷ hybridoma cells clone MAb using the Micro-Fast Track™ (Invitrogen, Carlsbad, Calif.). Complementary DNA was synthesized using a cDNA synthesis kit (Stratagene, La Jolla, Calif., USA) and cloned into the ZAP expression vector and packaged (Stratagene).

Two sets of degenerated primers were designed and synthesized (Quality Systems, Taiwan). The sequences are as follows: 1) mouse kappa light chain, including a region from framework (FR1) to constant region (CL),

ML-1 (forward): 5′-GGG AAT TCG A(C/T)A TTG TG(A/C) T(A/G)A C(A/C)C A(A/G)(G/T) (A/C)TC AA-3′ (SEQ ID NO:5),

ML-2 (reverse): 5′-GGA AGC TTA CTG GAT GGT GGG AAG ATG GA-3 (SEQ ID NO:6);

2) mouse gamma heavy chain, including a region from FR1 to C_{H}1,

MG-1 (forward): 5′-GGG AAT TC(G/C) AGG T(C/G)(A/C) A(A/G)C TGC AG(C/G) AGT CT-3′ (SEQ ID NO:7),

MG-2 (reverse): 5′-GCA AGC TTA (T/C)CT CCA CAC ACA GG(A/G) (A/G)CC AGT GGA TAG AC-3′ (SEQ ID NO:8).

EcoR I (GAATTC) and Hind III (AAGCTT) sites in the forward and reverse primers are underlined.

RT-PCR was performed as described above. Twenty-five nanograms of each RT-PCR product (light chain: 350 bp and heavy chain: 450 bp) was used to prepare DNA probe labeled with α-³²P-dCTP using a rediprime™ II DNA Labeling System (Amersham Pharmacia Biotech, Buckinghamshire, England.

Gene cloning and sequence analysis. cDNA prepared using the RT-PCR was cloned into EcoR I and Xho I sites of pBK-CMV phagemid vector and introduced into Escherichia coli XL1 Blue MRF' strain according to the method described in Chiu C. et al., Prot. Expr. Purif: 24: 292-301, 2002. Bacteria plaques were transferred onto Hybond-N nylon membranes (Amersham Pharmacia Biotech, Buckinghamshire, England). Bacteria DNA was denatured for 5 minutes in a denaturing solution (1.5 M NaCl, and 0.5 N NaOH) and neutralized in a neutralization solution (1 M Tris-HCl and 1.5 M NaCl; pH 7.4) for 5 minutes at room temperature. After affixing the DNA using a UV-cross linker (Model 1800, Stratagene, La Jolla, Calif., USA), the membranes were hybridized with the probe in a hybridization solution (250 mM sodium phosphate, pH 7.2, 50% formamide, 10% polyethylene glycol 8000, 250 mM NaCl, 0.5 mM EDTA and 7% SDS), according to the method described in Murali S. et al., J Fish Dis. 25: 91-100, 2002. The membranes were then washed in a solution containing 0.1×SSC and 0.1% SDS 3 times at 42° C. each for 30 minutes and air-dried before subjected to autoradiography. Randomly selected positive clones were cultured separately at 37° C. and used for sequence analyses. The nucleotide sequence of the insert in each clone was determined using the vector specific T3/T7 primers. The DNA sequencing was performed using Big Dye™ terminators (PE Biosystems, Foster City, Calif., USA) and analyzed on an ABI PRISM 377 DNA auto-sequencer. The full-length Ig cDNA sequences were compared with other Ig sequences using Kabat's database and the computer programs of the NCBI Gene Bank.

Isolation and cloning of RG-M18 heavy and light chain genes. cDNAs for the heavy and light chains of the RG-M18 monoclonal antibody described above (NNV-18H and NNV-18L) were cloned and sequenced as described above. The NNV-18H heavy chain cDNA contained 1620 bp, including a 5′ untranslated region (90 bp), a 3′ untranslated region (123 bp) and an open reading frame (1407 bp). The complete open reading frame was predicted to encode a protein of 469 amino acids (GenBank accession number: AF466698), including a signal sequence, three complementarity-determining regions (CDR1, CDR2, and CDR3), three constant regions (C_{H}1, C_{H}2 and C_{H}3), and a hinge region. The predicted molecular weight of the mature NNV-18H was 49.8 kDa. The NNV-18L light chain cDNA sequence (951 bp) included a 5′ untranslated region (30 bp), a 3′ untranslated region (219 bp) and an open reading frame (702 bp). The complete open reading frame was predicted to encode a protein of 234 amino acids (GenBank accession number: AF466699), including a signal sequence, CDR1, CDR2 and CDR3, and a constant region (CL). The predicted molecular weight of the mature NNV-18L protein was 23.6 kDa.

Vectors construction and transfection. The cDNAs encoding heavy (18H) and light (18L) chains were amplified by PCR using the primers, 18H-forward: 5′-TCA CCG CTA GCA TGA ACT TCG GGC TCA GC-3′ (SEQ ID NO:9), 18H-reverse, 5′-CTC AGC GGC CGC TCA TTT ACC CGG AGT CCG GGA 3′ (SEQ ID NO:10), 18L-forward: 5′-CAA GGC TAG CAT GGA GTC ACA GAC-3′ (SEQ ID NO:11), 18L-reverse: 5′-TCT AGC GGC CGC CTA ACA CTC ATT CCT GTT GAA-3′ (SEQ ID NO:12) (Nhe I and Not I sites in the forward and reverse primers are underlined). The PCR products were cloned (by replacing EGFP) into pEGFP—N1 (Clontech, Palo Alto, Calif.) expression vector, at Nhe I/Not I sites, to obtain the plasmids, pCMV-NNV-18H and pCMV-NNV-18L, respectively.

A bicistronic expression vector containing genes for both heavy and light chains was constructed as follows. First, the 18L gene and polyadenylation sequences from pCMV-NNV-18L were amplified by PCR using primers 18L-forward (SEQ ID NO:11) and 18L-PA: 5′-CCA AGC TTG CTC TAG AAG TAC TCT CGA GT-3′ (reverse, SEQ ID NO: 13) (Hind III site is underlined). To amplify the cytomegalovirus (CMV) promoter and 18H gene sequences from pCMV-NNV-18H, primers 18L-PR: 5′-TGC ATG GTA CCT AGT TAT TAA TAG TAA TC-3′ (forward, SEQ ID NO: 14) and 18H-reverse (SEQ ID NO:10), (Kpn I site is underlined) were used. The PCR reactions were performed using 20 ng of plasmid DNA. Following amplification and restriction enzymes digestion, the PCR products were subjected to 1% agarose gel electrophoresis, and eluted using a VIOGENE (Taipei, Taiwan) Gel Extraction Miniprep Kit. The 18L-poly(A) DNA fragment and CMV promoter-18H DNA fragment were sequentially cloned into pEGFP—N1 vector at Nhe I/Hind III and Kpn I/Not I sites, respectively. The resulting construct (pCMV-NNV-18HL) was confirmed by restriction endonuclease analysis and sequencing.

Expression of RG-M18 genes in GB cells. Three eukaryotic expression vectors, pCMV-NNV-18L containing the NNV-18L open reading frame, pCMV-NNV-18H containing the NNV-18H open reading frame, and bicistronic pCMV-NNV-18HL containing both open reading frame, were constructed as descried above. pCMV-NNV-18HL contains, an NNV-18L cDNA-poly(A) sequence (912 bp), a CMV-promoter sequence, and an NNV-18H cDNA sequence (1910 bp). The eDNAs encoding the light and heavy chains were under the control of independent CMV promoters.

GB cells (10⁵) were transfected with 10 μg of each the vectors using the LIPOFECTAMINE PLUS method (LIFE TECHNOLOGIES, Rockville, Md.). Stable transfected cells were selected in L-15 medium-10% FBS containing 1 mg/ml G418. G418-resistant cells were subcloned by limited dilution.

Intracellular expression of heavy and light chains of RG-M18 was examined using immunocytochemical staining. Transfected GB cells (10⁵) were grown in the Lab-Tek chamber slide™ system (4 well Permanox Slide, Nalge Nunc International Corp., IL, USA) at 28° C. overnight. After cooling the cell culture on ice and removing the media, the cells were washed with 4° C. PBS and fixed in 2% para-formaldehyde/0.1% Triton X-100 for 30 minutes on ice. Following removal of fixative and washing the cells twice in 4° C. PBS (5 minutes/wash), alkaline phosphatase-conjugated goat anti-mouse IgG Fe (Pierce, Rockford, Ill.) or anti-mouse IgG K chain (Cashmere Biotech, Taichung, Taiwan) antibody was added into chamber slides so that cells were just covered. The cells and antibodies were incubated at 4° C. for 60 minutes before the cells were washed for four times in 4° C. PBS (5 minutes/wash). Finally, the alkaline phosphatase was visualized using 5-bromo-4-chloro-3-indolyl phosphate and 4-nitro-blue tetrazolium chloride. The results revealed the expression of the mouse heavy and light chains in GB cells transfected with corresponding vectors, and no expression in cells transfected with a pEGFP—N1 control plasmid.

Secretion of assembled antibody by GB cells. The supernatant of the GB cells transfected with the vectors described above were analyzed using SDS-PAGE-Western blot as described above. GB cells transfected with pCMV-EGFP-N1, pCMV-NNV-18H, pCMV-NNV-18L, or pCMV-NNV-18HL were cultured in 24-well tissue culture plates for 3 days. One hundred microliter of supernatant from each well was harvested and mixed with an equal volume of SDS-PAGE 2×loading buffer (100 mM Tris-HCl, pH 6.8, 200 mM β-mercaptoethanol, 4% SDS, 0.2% bromophenol blue, and 20% glycerol). These mixtures were analyzed using Western blot as described above.

The heavy (about 50 kDa) and light (about 25 kDa) chains secreted by pCMV-NNV18HL-transfected GB cells were strongly recognized by the anti-mouse IgG Fc and anti-mouse IgG κ chain antibodies, respectively. In GB cells transfected with pCMV-NNV-18H or pCMV-NNV-18L, the heavy chains or the light chains were also recognized by corresponding antibodies.

Western blot was also performed under non-reducing condition, i.e., using a β-mercaptoethanol-free 2×loading buffer and without heating the samples. Under the non-reducing condition, only one major band with a molecular weight about 150 kDa was detected in the supernatant from pCMV-NNV-18HL-transfected GB cells. This band was recognized by both anti-mouse IgG Fe antibodies and anti-mouse IgG κ light chain antibodies. These results indicated that the heavy and light chains assembled, via disulfide bonds, to form the 150 kDa antibodies, which were secreted into the medium.

Since the antibodies had all four compete chains of naturally occurring antibodies, i.e., two of the heavy chain species and two of the light chain species, the antibodies could folded and assembly into the correct three-dimension structure. These correctly folded and assembled antibodies have long half-life. In contrast, the single chain antibodies mentioned early had short half-life due to lack of amino acids and disulfide bonds necessary for correct folding and assembly.

Detection and neutralizing activity of expressed antibodies. The neutralization efficiency of extracellular antibodies secreted by the GB cells transfected with pEGFP—N1 (control) and pCMV-NNV-18HL was carried-out as follows. 72 hours after transfection, 1 ml supernatant from each cell culture well was collected, mixed with an equal volume of 10 fold dilutions of YGNNV (titre 10⁸) in L-15 medium containing 2% FBS, and incubated at room temperature for 1 hour with gentle shaking. The mixture was then added into the 80% confluent monolayer of GB cells and incubated at room temperature for 1 hour. The supernatant was replaced with 1 ml of fresh L-15 medium containing 2% FBS. The culture plates were then incubated at 28° C. for 7 days and examined for the CPE. The neutralization titre was determined using the TCID₅₀ method as described above.

To examine the neutralization ability of the intracellular antibodies, cells transfected with pEGFP-N1 (control) or pCMV-NNV-18HL were seeded into 6-well dishes (10⁵ cells per well) and grown for overnight. The cells were infected with YGNNV at MOI of 100 as described above. Supernatant was collected at 6, 12 or 24 hours post infection, and was overlaid on 80% confluent monolayer of GB cells as described above. The supernatant was also subjected to Western blot analysis using antibody against the coat protein of YGNNV as described above. The neutralization index (NI) was calculated using Formula II:

NI=log₁₀(TCID₅₀ mL⁻¹ of the supernatant from control cells)−log₁₀(TCID₅₀ mL⁻¹ of the supernatant from transfected cells)

Binding and neutralizing activity of extra- and intracellular recombinant antibodies. The heavy or light chains secreted by the transfected GB cells could bind to YGNNV as revealed by the ELISA described above. The optical density values were 1, 0.6 and 0.5 for NNV-18HL, NNV-18H and NNV-18L respectively, indicating high efficiency of binding to the virus. In contrast, no binding activity was detected in the supernatant of control GB cells transfected with pEGFP—N1.

A remarkable reduction of the virus titre was observed after the virus was incubated with the supernatant of the GB cells transfected with pCMV-NNV-18HL. The log₁₀ (TCID₅₀ mL⁻¹) value of the supernatant of the GB cells was 4, while that of the control supernatant was 8. Accordingly, the neutralization index the supernatant of the GB cell was 4.

The cells expressing the antibodies were co-cultured with GB cells not transfected with the above-mentioned expression vectors. The cells mixture were inoculated with viruses and examined for the CPE as described before. The result revealed that the non-transfected cells surrounding the transfected cells were protected from the viruses.

The ability of intracelluar antibodies was examined by propagating the virus in the pCMV-NNV-18HL-transfected GB cells. Results indicated that the intracellular antibodies protected the cells from YGNNV. The log₁₀ (TCID₅₀ mL⁻) value of the supernatant from the cells was 6, and the neutralization index was 2. Western blot results also showed the considerable reduction in the secretion of YGNNV particles due to the efficient neutralization of intracellular antibodies.

### Other Embodiments

All of the features disclosed in this specification may be combined in any combination. Each feature disclosed in this specification may be replace by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A fish cell that expresses a mammalian protein complex having two peptide species, the cell comprising:
a first nucleic acid sequence encoding a first peptide species of the mammalian protein complex, and
a second nucleic acid sequence encoding a second peptide species of the mammalian protein complex, wherein a multiplicity of peptides of the first peptide species and the second peptide species are expressed and at least one peptide of the first peptide species and at least one peptide of the second peptide species assembly to form a functional mammalian protein complex.

2. The cell of claim 1, wherein the mammalian protein complex is an antibody.

3. The cell of claim 2 is a brain cell.

4. The cell of claim 2, wherein the antibody contains a disulfide bond.

5. The cell of claim 2, wherein the fish is a yellow grouper.

6. The cell of claim 5 is a brain cell.

7. The cell of claim 2, wherein the antibody binds to a microorganism.

8. The cell of claim 7, wherein the fish is a yellow grouper.

9. The cell of claim 7, wherein the microorganism is a virus.

10. The cell of claim 9, wherein the fish is a yellow grouper.

11. The cell of claim 9, wherein the virus is a nervous necrosis virus.

12. The cell of claim 11, wherein the fish is a yellow grouper.

13. The cell of claim 12, wherein the antibody binds to a coat protein of the virus.

14. The cell of claim 13 is a brain cell.

15. The cell of claim 1, wherein the mammalian protein complex contains a disulfide bond.

16. The cell of claim 15 is a brain cell.

17. The cell of claim 15, wherein the fish is a yellow grouper.

18. The cell of claim 17 is a brain cell.

19. The cell of claim 1, wherein the mammalian protein complex is a signaling molecule.

20. The cell of claim 19, wherein the signaling molecule contains a disulfide bond.

21. The cell of claim 20 is a brain cell.

22. The cell of claim 20, wherein the fish is a yellow grouper.

23. The cell of claim 22 is a brain cell.

24. A method of expressing in a fish a mammalian protein complex having two peptide species, the method comprising introducing into cells of the fish an eukaryotic expression cassette that contains two sequences encoding a first peptide species and a second peptide species, respectively, of the mammalian protein complex, each sequence being operably linked to a transcription promoter, whereby a multiplicity of peptides of the first peptide species and the second peptide species are expressed and at least one peptide of the first peptide species and at least one peptide of the second peptide species assembly to form the complex.

25. The method of claim 24, wherein the mammalian protein complex is an antibody.

26. The method of claim 25, wherein the antibody contains a disulfide bond.

27. The method of claim 25, wherein the fish is a yellow grouper.

28. The method of claim 27, wherein the cell is a brain cell.

29. The method of claim 25, wherein the cell is a brain cell.

30. The method of claim 25, wherein the antibody binds to a microorganism.

31. The method of claim 30, wherein the fish is a yellow grouper.

32. The method of claim 30, wherein the microorganism is a virus.

33. The method of claim 32, wherein the fish is a yellow grouper.

34. The method of claim 32, wherein the virus is a nervous necrosis virus.

35. The method of claim 34, wherein the fish is a yellow grouper.

36. The method of claim 35, wherein the antibody binds to a coat protein of the nervous necrosis virus.

37. The method of claim 36, wherein the cell is a brain cell.

38. The method of claim 24, wherein the mammalian protein complex contains a disulfide bond.

39. The method of claim 24, wherein the mammalian protein complex is a signaling molecule.

40. A cultured fish cell infected with a virus, or a progeny of the cell, wherein the cell produces the virus.

41. The cell of claim 40, wherein the virus is a yellow grouper nervous necrosis virus.

42. The cell of claim 40, wherein the cell is a brain cell.

43. The cell of claim 40, wherein the fish cell is a yellow grouper cell.


SEQUENCE LISTING (14 sequences)

SEQ ID NO: 1 - DNA, 33 bp, Artificial Sequence
  primer
ggaattccatatgggatttggacgtgcgaccaa

SEQ ID NO: 2 - DNA, 33 bp, Artificial Sequence
  primer
ggaattccatatgcgtgtcagtcatgtgtcgct

SEQ ID NO: 3 - DNA, 27 bp, Artificial Sequence
  primer
ccgctcgaggtttgcggggcacattgg

SEQ ID NO: 4 - DNA, 29 bp, Artificial Sequence
  primer
ccgctcgagcgagtcaacacgggtgaaga

SEQ ID NO: 5 - DNA, 32 bp, Artificial Sequence
  primer
gggaattcgayattgtgmtracmcarkmtcaa

SEQ ID NO: 6 - DNA, 29 bp, Artificial Sequence
  primer
ggaagcttactggatggtgggaagatgga

SEQ ID NO: 7 - DNA, 29 bp, Artificial Sequence
  primer
gggaattcsaggtsmarctgcagsagtct

SEQ ID NO: 8 - DNA, 38 bp, Artificial Sequence
  primer
gcaagcttayctccacacacaggrrccagtggatagac

SEQ ID NO: 9 - DNA, 29 bp, Artificial Sequence
  primer
tcaccgctagcatgaacttcgggctcagc

SEQ ID NO: 10 - DNA, 33 bp, Artificial Sequence
  primer
ctcagcggccgctcatttacccggagtccggga

SEQ ID NO: 11 - DNA, 24 bp, Artificial Sequence
  primer
caaggctagcatggagtcacagac

SEQ ID NO: 12 - DNA, 33 bp, Artificial Sequence
  primer
tctagcggccgcctaacactcattcctgttgaa

SEQ ID NO: 13 - DNA, 29 bp, Artificial Sequence
  primer
ccaagcttgctctagaagtactctcgagt

SEQ ID NO: 14 - DNA, 29 bp, Artificial Sequence
  primer
tgcatggtacctagttattaatagtaatc